(19) United States
(12) Patent Application Publication
(10) Pub. No.: US 2022/0224876 A1
(43) Pub. Date: Jul. 14, 2022

(54) Dermatological Imaging Systems and Methods for Generating Three-Dimensional (3D) Image Models

(71) Applicants: The Procter & Gamble Company, Cincinnati, OH (US); Canfield Scientific, Inc., Parsippany, NJ (US)

(72) Inventors: Paul Jonathan MATTS, Addlestone (GB); Mani V. THOMAS, Ringoes, NJ (US); Daniel Eric DiGREGORIO, Fairfield, NJ (US)

(21) Appl. No.: 17/572,709

(22) Filed: Jan. 11, 2022

Related U.S. Application Data
(60) Provisional application No. 63/136,066, filed on Jan. 11, 2021.

(51) Int. Cl. H04N 13/254; G06T 17/00; G06T 7/00; H04N 13/296; G16H 30/40; G16H 40/40

(52) U.S. Cl. CPC H04N 13/254; G06T 17/00; G06T 7/0014; G06T 2207/30088; G16H 30/40; G16H 40/40; H04N 13/296

(57) ABSTRACT

Systems and methods are described for generating three-dimensional (3D) image models of skin surfaces. An example method includes analyzing, by one or more processors, a plurality of images of a portion of skin of a user, the plurality of images captured by a camera having an imaging axis extending through one or more lenses configured to focus the portion of skin, wherein each image of the plurality of images is illuminated by a different subset of a plurality of LEDs configured to be positioned at a perimeter of the portion of skin. The example method may further include generating, by the one or more processors, a 3D image model defining a topographic representation of the portion of skin based on the plurality of images; and generating, by the one or more processors, a user-specific recommendation based on the 3D image model of the portion of skin.

### FIELD OF THE INVENTION

The present disclosure generally relates to dermatological imaging systems and methods, and more particularly to, dermatological imaging systems and methods for generating three-dimensional (3D) image models.

### BACKGROUND OF THE INVENTION

Skin health, and correspondingly, skin care plays a vital role in the overall health and appearance of all people. Many common activities have an adverse effect on skin health, so a well-informed skin care routine and regular visits to a dermatologist for evaluation and diagnosis of any skin conditions is a priority for millions. Problematically, scheduling dermatologist visits can be cumbersome, time consuming, and may put the patient at risk of a skin condition worsening if a prompt appointment cannot be obtained. Moreover, conventional dermatological methods for evaluating many common skin conditions can be inaccurate, such as by failing to accurately and reliably identify abnormal textures or features on the skin surface.

As a result, many patients may neglect receiving regular dermatological evaluations, and may further neglect skin care altogether from a general lack of understanding. The problem is acutely pronounced given the myriad of skin conditions that may develop, and the associated myriad of products and treatment regimens available. Such existing skin care products may also provide little or no feedback or guidance to assist the user in determining whether or not the product applies to their skin condition, or how best to utilize the product to treat the skin condition. Thus, many patients purchase incorrect or unnecessary products to treat or otherwise manage a real or perceived skin condition because they incorrectly diagnose a skin condition or fail to purchase products that would effectively treat the skin condition.

For the foregoing reasons, there is a need for dermatological imaging systems and methods for generating three-dimensional (3D) image models of skin surfaces.

### SUMMARY OF THE INVENTION

Described herein is a dermatological imaging system configured to generate 3D image models of skin surfaces. The dermatological imaging system includes a dermatological imaging device comprising a plurality of light-emitting diodes (LEDs) configured to be positioned at a perimeter of a portion of skin of a user, and one or more lenses configured to focus the portion of skin. The dermatological imaging system further includes a computer application (app) comprising computing instructions that, when executed on a processor, cause the processor to: analyze a plurality of images of the portion of skin, the plurality of images captured by a camera having an imaging axis extending through the one or more lenses, wherein each image of the plurality of images is illuminated by a different subset of the plurality of LEDs, generate, based on the plurality of images, a 3D image model defining a topographic representation of the portion of skin. A user-specific recommendation can be generated based on the 3D image model of the portion of skin.

The dermatological imaging system described herein includes improvements to other technologies or technical fields at least because the present disclosure describes or introduces improvements to the field of dermatological imaging devices and accompanying skin care products. For example, the dermatological imaging device of the present disclosure enables a user to quickly and conveniently capture skin surface images and receive a complete 3D image model of the imaged skin surface on a display of a user's mobile device. In addition, the dermatological imaging system includes specific features other than what is well-understood, routine, conventional activity in the field, or adding unconventional steps that confine the claim to a particular useful application, e.g., capturing skin surface images for analysis using an imaging device in contact with the skin surface where the camera is disposed a short imaging distance from the skin surface.

The dermatological imaging system herein provides improvements in computer functionality or in improvements to other technologies at least because the improving the intelligence or predictive ability of a user computing device with a trained 3D image modeling algorithm. The 3D image modeling algorithm, executing on the user computing device or imaging server, is able to accurately generate, based on pixel data of the user's portion of skin, a 3D image model defining a topographic representation of the users' portion of skin. The 3D image modeling algorithm also generates a user-specific recommendation (e.g., for a manufactured product or medical attention) designed to address a feature identifiable within the pixel data of the 3D image model. This is in improvement over conventional systems at least because conventional systems lack such real-time generative or classification functionality and are simply not capable of accurately analyzing user-specific images to output a user-specific result to address a feature identifiable within the pixel data of the 3D image model.

### BRIEF DESCRIPTION OF THE DRAWINGS

FIG. 1 illustrates an example of a digital imaging system.

FIG. 2A is an overhead view of an imaging device;

FIG. 2B is a cross-sectional side view along axis-2B of the imaging device of FIG. 2A.

FIG. 2C is an enlarged view of the portion indicated in FIG. 2B.

FIG. 3A illustrates a camera calibration surface used to calibrate a camera.

FIG. 3B is an illumination calibration diagram.

FIG. 4 illustrates an example video sampling period that may be used to synchronize the camera image captures with an illumination sequence.

FIG. 5A illustrates an example image and its related pixel data that may be used for training and/or implementing a 3D image modeling algorithm.

FIG. 5B illustrates an example image and its related pixel data that may be used for training and/or implementing a 3D image modeling algorithm.

FIG. 5C illustrates an example image and its related pixel data that may be used for training and/or implementing a 3D image modeling algorithm.

FIG. 6 illustrates an example workflow of a 3D image modeling algorithm using an input skin surface image to generate a 3D image model defining a topographic representation of the skin surface.

FIG. 7 illustrates a diagram of an imaging method for generating 3D image models of skin surfaces.

FIG. 8 illustrates an example user interface as rendered on a display screen of a user computing device.

### DETAILED DESCRIPTION OF THE INVENTION

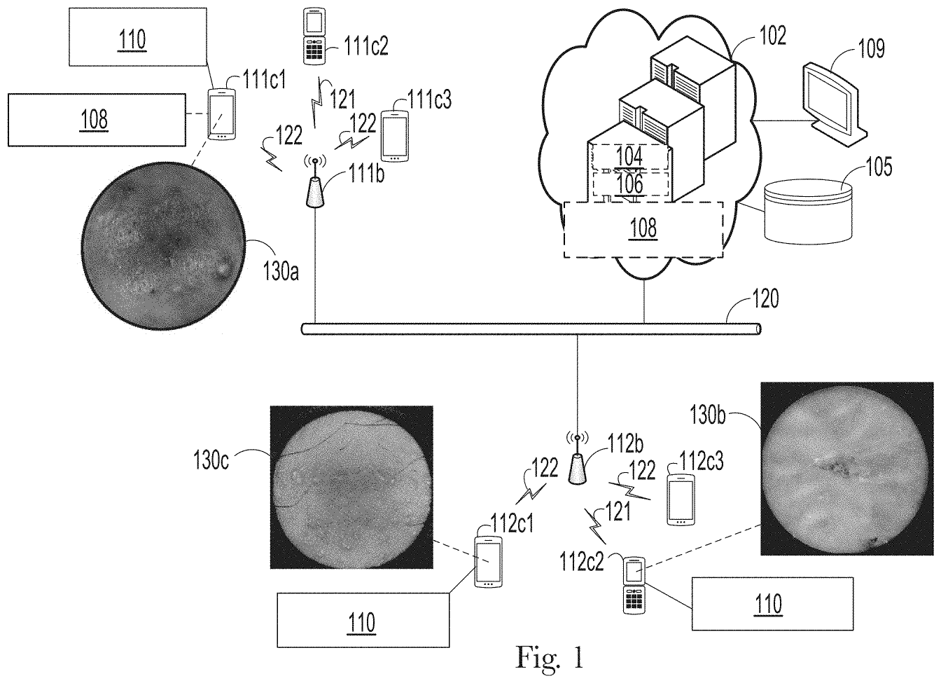

FIG. 1 illustrates an example digital imaging system 100 configured to analyze pixel data of an image (e.g., image(s) 130a, 130b, and/or 130c) of a user's skin surface for generating a 3D image model of the user's skin surface, in accordance with various embodiments disclosed herein. As referred to herein, a “skin surface” may refer to any portion of the human body including the torso, waist, face, head, arm, leg, or other appendage or portion or part of the user's body thereof. In the example embodiment of FIG. 1, digital imaging system 100 includes imaging server(s) 102 (also referenced herein as “server(s)”), which may comprise one or more computer servers. In various embodiments imaging server(s) 102 comprise multiple servers, which may comprise a multiple, redundant, or replicated servers as part of a server farm. In still further embodiments, imaging server(s) 102 may be implemented as cloud-based servers, such as a cloud-based computing platform. For example, server(s) 102 may be any one or more cloud-based platform(s) such as MICROSOFT AZURE, AMAZON AWS, or the like. Server(s) 102 may include one or more processor(s) 104 as well as one or more computer memories 106.

The memories 106 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memorie(s) 106 may store an operating system (OS) (e.g., Microsoft Windows, Linux, Unix, etc.) capable of facilitating the functionalities, apps, methods, or other software as discussed herein. The memorie(s) 106 may also store a 3D image modeling algorithm 108, which may be an artificial intelligence based model, such as a machine learning model trained on various images (e.g., image(s) 130a, 130b, and/or 130c), as described herein. Additionally, or alternatively, the 3D image modeling algorithm 108 may also be stored in database 105, which is accessible or otherwise communicatively coupled to imaging server(s) 102, and/or in the memorie(s) of one or more user computing devices 111c1-111c3 and/or 112c1-112c3. The memories 106 may also store machine readable instructions, including any of one or more application(s), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For example, at least some of the applications, software components, or APIs may be, include, otherwise be part of, an imaging-based machine learning model or component, such as the 3D image modeling algorithm 108, where each may be configured to facilitate their various functionalities discussed herein. It should be appreciated that one or more other applications may be envisioned and that are executed by the processor(s) 104.

The processor(s) 104 may be connected to the memories 106 via a computer bus responsible for transmitting electronic data, data packets, or otherwise electronic signals to and from the processor(s) 104 and memories 106 in order to implement or perform the machine-readable instructions, methods, processes, elements or limitations, as illustrated, depicted, or described for the various flowcharts, illustrations, diagrams, figures, and/or other disclosure herein.

The processor(s) 104 may interface with the memory 106 via the computer bus to execute the operating system (OS). The processor(s) 104 may also interface with the memory 106 via the computer bus to create, read, update, delete, or otherwise access or interact with the data stored in the memories 106 and/or the database 104 (e.g., a relational database, such as Oracle, DB2, MySQL, or a NoSQL based database, such as MongoDB). The data stored in the memories 106 and/or the database 105 may include all or part of any of the data or information described herein, including, for example, training images and/or user images (e.g., either of which including any image(s) 130a, 130b, and/or 130c) or other information of the user, including demographic, age, race, skin type, or the like.

The imaging server(s) 102 may further include a communication component configured to communicate (e.g., send and receive) data via one or more external/network port(s) to one or more networks or local terminals, such as computer network 120 and/or terminal 109 (for rendering or visualizing) described herein. In some embodiments, imaging server(s) 102 may include a client-server platform technology such as ASP.NET, Java J2EE, Ruby on Rails, Node.js, a web service or online API, responsive for receiving and responding to electronic requests. The imaging server(s) 102 may implement the client-server platform technology that may interact, via the computer bus, with the memories(s) 106 (including the applications(s), component(s), API(s), data, etc. stored therein) and/or database 105 to implement or perform the machine-readable instructions, methods, processes, elements or limitations, as illustrated, depicted, or described for the various flowcharts, illustrations, diagrams, figures, and/or other disclosure herein. According to some embodiments, the imaging server(s) 102 may include, or interact with, one or more transceivers (e.g., WWAN, WLAN, and/or WPAN transceivers) functioning in accordance with IEEE standards, 3GPP standards, or other standards, and that may be used in receipt and transmission of data via external/network ports connected to computer network 120. In some embodiments, computer network 120 may comprise a private network or local area network (LAN). Additionally, or alternatively, computer network 120 may comprise a public network such as the Internet.

Imaging server(s) 102 may further include or implement an operator interface configured to present information to an administrator or operator and/or receive inputs from the administrator or operator. As shown in FIG. 1, an operator interface may provide a display screen (e.g., via terminal 109). Imaging server(s) 102 may also provide I/O components (e.g., ports, capacitive or resistive touch sensitive input panels, keys, buttons, lights, LEDs), which may be directly accessible via or attached to imaging server(s) 102 or may be indirectly accessible via or attached to terminal 109. According to some embodiments, an administrator or operator may access the server 102 via terminal 109 to review information, make changes, input training data or images, and/or perform other functions.

As described above herein, in some embodiments, imaging server(s) 102 may perform the functionalities as discussed herein as part of a “cloud” network or may otherwise communicate with other hardware or software components within the cloud to send, retrieve, or otherwise analyze data or information described herein.

In general, a computer program or computer based product, application, or code (e.g., the model(s), such as AI models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 104 (e.g., working in connection with the respective operating system in memories 106) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

As shown in FIG. 1, imaging server(s) 102 are communicatively connected, via computer network 120 to the one or more user computing devices 111c1-111c3 and/or 112c1-112c3 via base stations 111b and 112b. In some embodiments, base stations 111b and 112b may comprise cellular base stations, such as cell towers, communicating to the one or more user computing devices 111c1-111c3 and 112c1-112c3 via wireless communications 121 based on any one or more of various mobile phone standards, including NMT, GSM, CDMA, UMMTS, LTE, 5G, or the like. Additionally or alternatively, base stations 111b and 112b may comprise routers, wireless switches, or other such wireless connection points communicating to the one or more user computing devices 111c1-111c3 and 112c1-112c3 via wireless communications 122 based on any one or more of various wireless standards, including by non-limiting example, IEEE 802.11a/b/c/g (WIFI), the BLUETOOTH standard, or the like.

Any of the one or more user computing devices 111c1-111c3 and/or 112c1-112c3 may comprise mobile devices and/or client devices for accessing and/or communications with imaging server(s) 102. In various embodiments, user computing devices 111c1-111c3 and/or 112c1-112c3 may comprise a cellular phone, a mobile phone, a tablet device, a personal data assistance (PDA), or the like, including, by non-limiting example, an APPLE iPhone or iPad device or a GOOGLE ANDROID based mobile phone or tablet. In still further embodiments, user computing devices 111c1-111c3 and/or 112c1-112c3 may comprise a home assistant device and/or personal assistant device, e.g., having display screens, including, by way of non-limiting example, any one or more of a GOOGLE HOME device, an AMAZON ALEXA device, an ECHO SHOW device, or the like.

Further, the user computing devices 111c1-111c3 and/or 112c1-112c3 may comprise a retail computing device, configured in the same or similar manner, e.g., as described herein for user computing devices 111c1-111c3. The retail computing device(s) may include a processor and memory, for implementing, or communicating with (e.g., via server(s) 102), a 3D image modeling algorithm 108 as described herein. However, a retail computing device may be located, installed, or otherwise positioned within a retail environment to allow users and/or customers of the retail environment to utilize the digital imaging systems and methods on site within the retail environment. For example, the retail computing device may be installed within a kiosk for access by a user. The user may then upload or transfer images (e.g., from a user mobile device) to the kiosk to implement the dermatological imaging systems and methods described herein. Additionally or alternatively, the kiosk may be configured with a camera and the dermatological imaging device 110 to allow the user to take new images (e.g., in a private manner where warranted) of himself or herself for upload and analysis. In such embodiments, the user or consumer himself or herself would be able to use the retail computing device to receive and/or have rendered a user-specific recommendation, as described herein, on a display screen of the retail computing device. Additionally or alternatively, the retail computing device may be a mobile device (as described herein) as carried by an employee or other personnel of the retail environment for interacting with users or consumers on site. In such embodiments, a user or consumer may be able to interact with an employee or otherwise personnel of the retail environment, via the retail computing device (e.g., by transferring images from a mobile device of the user to the retail computing device or by capturing new images by a camera of the retail computing device focused through the dermatological imaging device 110), to receive and/or have rendered a user-specific recommendation, as described herein, on a display screen of the retail computing device.

In addition, the one or more user computing devices 111c1-111c3 and/or 112c1-112c3 may implement or execute an operating system (OS) or mobile platform such as Apple's iOS and/or Google's Android operation system. Any of the one or more user computing devices 111c1-111c3 and/or 112c1-112c3 may comprise one or more processors and/or one or more memories for storing, implementing, or executing computing instructions or code, e.g., a mobile application or a home or personal assistant application, configured to perform some or all of the functions of the present disclosure, as described in various embodiments herein. As shown in FIG. 1, the 3D image modeling algorithm 108 may be stored locally on a memory of a user computing device (e.g., user computing device 111c1). Further, the mobile application stored on the user computing devices 111c1-111c3 and/or 112c1-112c3 may utilize the 3D image modeling algorithm 108 to perform some or all of the functions of the present disclosure.

In addition, the one or more user computing devices 111c1-111c3 and/or 112c1-112c3 may include a digital camera and/or digital video camera for capturing or taking digital images and/or frames (e.g., which can be image(s) 130a, 130b, and/or 130c). Each digital image may comprise pixel data for training or implementing model(s), such as artificial intelligence (AI), machine learning models, and/or rule-based algorithms, as described herein. For example, a digital camera and/or digital video camera of, e.g., any of user computing devices 111c1-111c3 and/or 112c1-112c3 may be configured to take, capture, or otherwise generate digital images and, at least in some embodiments, may store such images in a memory of a respective user computing devices. A user may also attach the dermatological imaging device 110 to a user computing device to facilitate capturing images sufficient for the user computing device to locally process the captured images using the 3D image modeling algorithm 108.

Still further, each of the one or more user computing devices 111c1-111c3 and/or 112c1-112c3 may include a display screen for displaying graphics, images, text, product recommendations, data, pixels, features, and/or other such visualizations or information as described herein. These graphics, images, text, product recommendations, data, pixels, features, and/or other such visualizations or information may be generated, for example, by the user computing device as a result of implementing the 3D image modeling algorithm 108 utilizing images captured by a camera of the user computing device focused through the dermatological imaging device 110. In various embodiments, graphics, images, text, product recommendations, data, pixels, features, and/or other such visualizations or information may be received by server(s) 102 for display on the display screen of any one or more of user computing devices 111c1-111c3 and/or 112c1-112c3. Additionally or alternatively, a user computing device may comprise, implement, have access to, render, or otherwise expose, at least in part, an interface or a guided user interface (GUI) for displaying text and/or images on its display screen.

User computing devices 111c1-111c3 and/or 112c1-112c3 may comprise a wireless transceiver to receive and transmit wireless communications 121 and/or 122 to and from base stations 111b and/or 112b. Pixel based images (e.g., image(s) 130a, 130b, and/or 130c) may be transmitted via computer network 120 to imaging server(s) 102 for training of model(s) and/or imaging analysis as described herein.

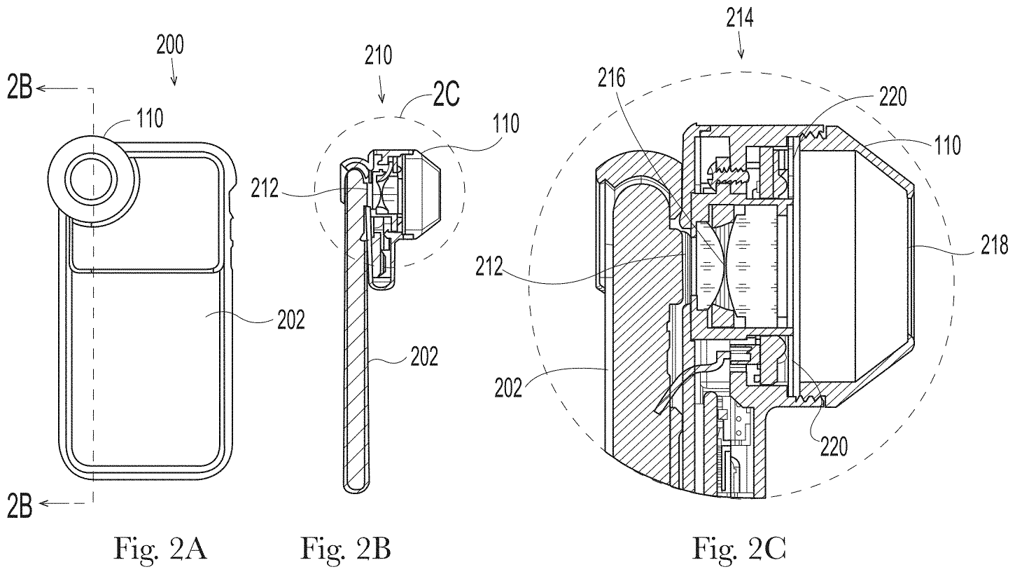

FIG. 2 is an overhead view 200, a side view 210, and a cutaway view 214 of a dermatological imaging device 110, in accordance with various embodiments disclosed herein. The overhead view 200 features the dermatological imaging device 110 attached to the back portion of a user mobile device 202. Generally, the dermatological imaging device 110 is configured to couple to the user mobile device 202 in a manner that positions the camera of the user mobile device in optical alignment with the lens and aperture of the dermatological imaging device 110. It is to be appreciated that the dermatological imaging device 110 may detachably or immovably couple to the user mobile device 202 using any suitable means.

The side view 210 illustrates the position of the dermatological imaging device 110 with respect to the camera 212 of the user mobile device 202. More specifically, the cutaway view 214 illustrates the alignment of the camera 212 of the user mobile device 202 with the lens set 216 and the aperture 218 of the dermatological imaging device 110. The lens set 216 may be configured to focus the camera 212 on objects positioned at a distance of the aperture 218 from the camera 212. Thus, as discussed further herein, a user may place the aperture of the dermatological imaging device 110 in contact with a portion of the user's skin, and the lens set 216 will enable the camera 212 of the user mobile device 202 to capture an image of the user's skin portion. In various embodiments, the distance from the aperture 218 to the camera 212 may define a short imaging distance, which may be less than or equal to 35 mm In various embodiments, the aperture 218 may be circular, and may have a diameter of approximately 20 mm.

The dermatological imaging device 110 may also include light-emitting diodes (LEDs) 220 configured to illuminate objects placed within the field of view (FOV) of the camera 212 through the aperture 218. Each of the LEDs 220 may be positioned within the dermatological imaging device 110, and may be arranged within the dermatological imaging device 110 such that the LEDs 220 form a perimeter around objects placed within the FOV defined by the aperture 218. For example, a user may place the user mobile device 202 and dermatological imaging device 110 combination on a portion of the user's skin so that the portion of skin is visible to the camera 212 through the aperture 218. The LEDs 220 may be positioned within the dermatological imaging device 110 in a manner that forms a perimeter around the portion of skin. Moreover, the dermatological imaging device 110 may include any suitable number of LEDs 220. In various embodiments, the dermatological imaging device 110 may include 21 LEDs 220, and they may be evenly distributed in an approximately circular, ring-like fashion to establish the perimeter around objects placed within the FOV defined by the aperture 218. In some embodiments, the LEDs 220 may be positioned between the camera 212 and the aperture 218 at approximately half the distance from the camera 212 to the aperture 218.

At such short imaging distances, conventional imaging systems may suffer from substantial internal reflection of a light source, resulting in poor image quality. To avoid these issues of conventional imaging systems, the inner surface 222 of the dermatological imaging device 110 may be coated with a high light absorptivity paint. In this manner, the LEDs 220 may illuminate objects in contact with an exterior surface of the aperture 218 without creating substantial internal reflections, thereby ensuring optimal image quality.

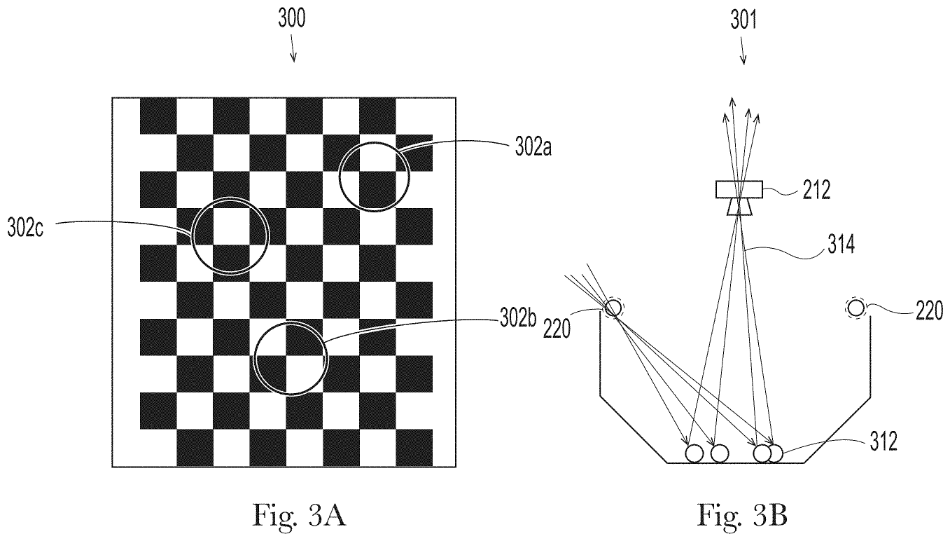

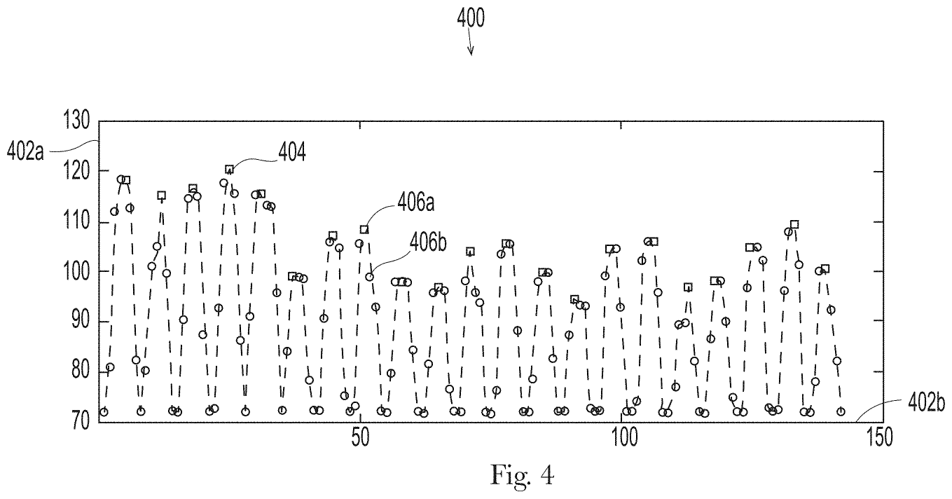

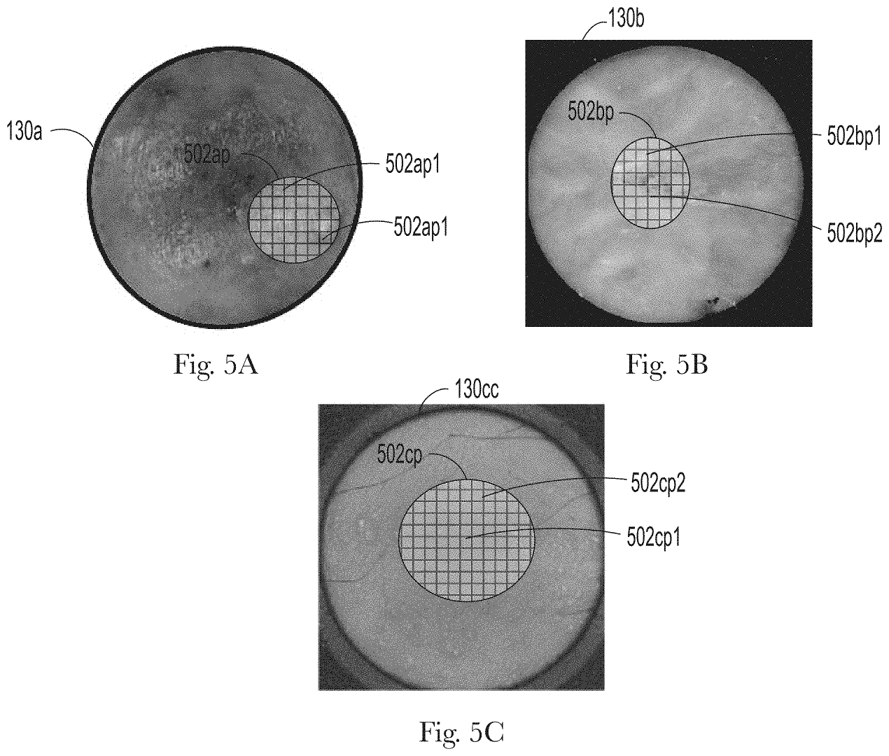

However, to further ensure optimal image quality and that the 3D image modeling algorithm may optimally perform the functions described herein, the camera 212 and LEDs 220 may be calibrated. Conventional systems may struggle to calibrate cameras and illumination devices at such short imaging distances due to distorted image characteristics (e.g., object surface degradation), and other similar abnormalities. The techniques of the present disclosure solve these problems associated with conventional systems using, for example, a random sampling consensus algorithm (discussed with respect to FIG. 3A) and light ray path tracing (discussed with respect to FIG. 3B). More generally, each of FIGS. 3A, 3B, and 4 describe calibration techniques that may be used to overcome the shortcomings of conventional systems, and that may be performed prior to, or as part of, the 3D image modeling techniques described herein in reference to FIGS. 5A-8.

FIG. 3A illustrates an example camera calibration surface 300 used to calibrate a camera (e.g., camera 202) for use with the dermatological imaging device 110 of FIGS. 2A-2C, and in accordance with various embodiments disclosed herein. Generally, the example camera calibration surface 300 may have known dimensions and may include a pattern or other design used to divide the example camera calibration surface 300 into equally spaced/dimensioned sub-sections. As illustrated in FIG. 3A, the example camera calibration surface 300 includes a checkerboard pattern, and each square of the pattern may have equal dimensions. Using image data derived from images captured of the example camera calibration surface 300, the user mobile device 202 may determine imaging parameters corresponding to the camera 212 and lens set 216. The image data may broadly refer to dimensions of identifiable features represented in an image of the example camera calibration surface 300. For example, the user mobile device 202 may determine (e.g., via a mobile application) scaling parameters that apply to images captured by the camera 212 when the dermatological imaging device 110 is attached to the user mobile device 202, a focal length, a distance to the focal plane, and/or other suitable parameters based on the image data derived from the images of the example camera calibration surface 300.

To begin calibrating the camera 212, a user may place the user mobile device 202 and dermatological imaging device 110 combination over the example camera calibration surface 300. When the user mobile device 202 and dermatological imaging device 110 are in position, the user mobile device 202 may prompt a user to perform a calibration image capture sequence and/or the user may manually commence the calibration image capture sequence. The user mobile device 202 may proceed to capture one or more images of the example camera calibration surface 300, and the user may slide or otherwise move the user mobile device 202 and dermatological imaging device 110 combination across the example camera calibration surface 300 to capture images of different portions of the surface 300. In some embodiments, the calibration image capture sequence is a video sequence, and the user mobile device 202 may analyze still frames from the video sequence to derive the image data. In other embodiments, the calibration image capture sequence is a series of single image captures, and the user mobile device 202 may prompt a user between each capture to move the user mobile device 202 and dermatological imaging device 110 combination to a different location on the example camera calibration surface 300.

During (e.g., in real-time) or after the calibration image capture sequence, the user mobile device 202 may select a set of images from the video sequence or series of single image captures to determine the image data. Generally, each image in the set of images may feature ideal imaging characteristics suitable to determine the image data. For example, the user mobile device 202 may select images representing or containing each of the regions 302a, 302b, and 302c by using a random sampling consensus algorithm configured to identify such regions based upon their image characteristics. The images containing these regions 302a, 302b, 302c may include an optimal contrast between the differently colored/patterned squares of the checkerboard pattern, minimal image degradation (e.g., resolution interference) due to physical effects associated with moving the user mobile device 202 and dermatological imaging device 110 combination across the example camera calibration surface 300, and/or any other suitable imaging characteristics or combinations thereof.

Using each image in the set of images, the user mobile device 202 (e.g., via the mobile app) may determine the image data by, for example, correlating identified image features with known feature dimensions. A single square within the checkerboard pattern of the example camera calibration surface 300 may measure 10 mm by 10 mm Thus, if the user mobile device 202 identifies that the image representing region 302c includes one full square, the user mobile device 202 may correlate the region within the image to measure 10 mm by 10 mm. This image data may also be compared to the known dimensions of the dermatological imaging device 110. For example, the aperture 218 of the dermatological imaging device 110 may measure 20 mm in diameter, such that areas represented by images captured by the camera 212 when the user mobile device 202 and dermatological imaging device 110 combination is in contact with a surface may generally not measure more than 20 mm in diameter. Accordingly, the user mobile device 202 may more accurately determine the image data in view of the approximate dimensions of the area represented by the image. Of course, surface abnormalities or other defects may cause the area represented by the image to be greater than the known dimensions of the aperture 218. For example, a user may press the dermatological imaging device 110 into a flexible surface (e.g., a skin surface) using sufficient force to distort the surface, causing a larger amount of the surface area to enter the dermatological imaging device 110 through the aperture 218 than a circular area defined by a 20 mm diameter.

In any event, the LEDs 220 may also require calibration to optimally perform the 3D image modeling functions described herein. FIG. 3B is an illumination calibration diagram 310 corresponding to an example calibration technique for illumination components (e.g., the LEDs 220) of the dermatological imaging device 110 of FIGS. 2A-2C, and in accordance with various embodiments disclosed herein. The illumination calibration diagram 310 includes the camera 212, multiple LEDs 220 illuminating objects 312, and light rays 314 representing paths the illumination emitted from the LEDs 220 traversed to reach the camera 212. The user mobile device 202 (e.g., via the mobile application) may initiate an illumination calibration sequence in which each of the LEDs 220 within the dermatological imaging device 110 individually ramps up/down to illuminate the objects 312, and the camera 212 captures an image corresponding to each respective LED 220 individually illuminating the objects 312. The objects 312 may be, for example, ball bearings and/or any other suitable objects or combinations thereof.

As illustrated in FIG. 3B, the illumination emitted from the left-most LED 220 is incident on each of the objects 312 and reflects up to the camera 212 along the paths represented by the light rays 314. The user mobile device 202 may include, as part of the mobile application, a path tracing module configured to trace each of the light rays reflected from the objects 312 back to their point of intersection. In doing so, the path tracing module may identify the location of the left-most LED 220. Accordingly, the user mobile device 202 may calculate the 3D position and direction corresponding to each of the LEDs 220 and their respective illumination, along with, for example, the number of LEDs 220, an illumination angle associated with each respective LED 220, an intensity of each respective LED 220, a temperature of the illumination emitted from each respective LED 220, and/or any other suitable illumination parameter. The illumination calibration diagram 310 includes four objects 312, and the user mobile device 202 may require at least two objects 312 reflecting illumination from the LEDs 220 to accurately identify a point of intersection, thereby enabling the illumination calibration sequence.

Advantageously, with the camera 212 and the LEDs 220 properly calibrated, the user mobile device 202 and dermatological imaging device 110 combination may perform the 3D image modeling functionality described herein. However, other physical effects (e.g., camera jitter) may further frustrate the 3D image modeling functionality despite the calibrations. To minimize the impact of these other physical effects the camera 212 and the LEDs 220 may be controlled asynchronously. Such asynchronous control may prevent the surface being imaged from moving during an image capture, and as a result, may minimize the impact of effects like camera jitter. As part of the asynchronous control, the camera 212 may perform a video sampling period in which the camera 212 captures a series of frames (e.g., high-definition (HD) video) while each LED 220 independently ramps up/down in an illumination sequence.

Generally, asynchronous control of the camera 212 and the LEDs 220 may result in frames captured by the camera 212 as part of the video sampling period that do not feature a respective LED 220 fully ramped up (e.g., fully illuminated). To resolve this potential issue, the user mobile device 202 may include a synchronization module (e.g., as part of the mobile application) configured to synchronize the camera 212 frames with the LED 220 ramp up times by identifying individual frames that correspond to fully ramped up LED 220 illumination. FIG. 4 is a graph 400 illustrating an example video sampling period the synchronization module may use to synchronize the camera 212 frame captures with an illumination sequence of the illumination components (e.g., the LEDs 220) of the dermatological imaging device 110 of FIGS. 2A-2C, and in accordance with various embodiments disclosed herein. The graph 400 includes an x-axis that corresponds to individual frames captured by the camera 212 and a y-axis that corresponds to the mean pixel intensity of a respective frame. Each circle (e.g., frame capture 404, 406a, 406b) included in the graph corresponds to a single image capture by the camera 212, and some of the circles (e.g., frame capture 404, 406a) additionally include a square circumscribing the circle indicating that the image capture represented by the circumscribed circle has a maximum mean pixel intensity corresponding to emitted illumination of an individual LED 220.

As illustrated in FIG. 4, the graph 400 has twenty-one peaks, each peak corresponding to a ramp up/down sequence of a particular LED 220. The user mobile device 202 (e.g., via the mobile application) may asynchronously initiate a video sampling period and an illumination sequence, such that the camera 212 may capture HD video during the video sampling period of each LED 220 individually ramping up/down to illuminate the region of interest (ROI) visible through the aperture 218, as part of the illumination sequence. As a result, the camera 212 may capture multiple frames of the ROI that include illumination from one or more LEDs 220 while partially and/or fully illuminated. The synchronization module may analyze each frame to generate a plot similar to the graph 400, featuring the mean pixel intensity of each captured frame, and may further determine frame captures corresponding to a maximum mean pixel intensity for each LED 220. The synchronization module may, for example, use a predetermined number of LEDs 220 to determine the number of maximum mean pixel intensity frame captures, and/or the module may determine a number of peaks included in the generated plot.

To illustrate, the synchronization module may analyze the pixel intensity of the first seven captured frames based on a known ramp up time for each LED 220 (e.g., a ramp up/down frame bandwidth), determine a maximum mean pixel intensity value among the first seven frames, designate the frame corresponding to the maximum mean pixel intensity as an LED 220 illuminated frame, and proceed to analyze the subsequent seven captured frames in a similar fashion until all captured frames are analyzed. Additionally or alternatively, the synchronization module may continue to analyze captured frames until a number of frames are designated as maximum mean pixel intensity frames corresponding to the predetermined number of LEDs 220. For example, if the predetermined number of LEDs 220 is twenty-one, the synchronization module may continue analyzing captured frames until twenty-one captured frames are designated as maximum mean pixel intensity frames.

Of course, the pixel intensity values may be analyzed according to a mean pixel intensity, an average pixel intensity, a weighted average pixel intensity, and/or any other suitable pixel intensity measurement or combinations thereof. Moreover, the pixel intensity may be computed in a modified color space (e.g., different color space than a red-green-blue (RGB) space). In this manner, the signal profile of the pixel intensity within the ROI may be improved, and as a result, the synchronization module may more accurately designate/determine maximum mean pixel intensity frames.

Once the synchronization module designates a maximum mean pixel intensity frame corresponding to each LED 220, the synchronization module may automatically identify frames containing full illumination from each respective LED 220 in subsequent video sampling periods captured by the user mobile device 202 and dermatological imaging device 110 combination. Each video sampling period may span the same number of frame captures, and the asynchronous control of the LEDs 220 may cause each LED 220 to ramp up/down in the same frames of the video sampling period and in the same sequential firing order. Thus, after a particular video sampling period, the synchronization module may automatically designate frame captures 404 406a as maximum mean pixel intensity frames, and may automatically designate frame capture 406b as a non-maximum mean pixel intensity frame. It will be appreciated that the synchronization module may perform the synchronization techniques described herein once to initially calibrate (e.g., synchronize) the video sampling period and illumination sequence, multiple times according to a predetermined frequency or as determined in real-time to periodically re-calibrate the video sampling period and illumination sequence, and/or as part of each video sampling period and illumination sequence.

When the user mobile device 202 and dermatological imaging device 110 combination is properly calibrated, a user may begin capturing images of their skin surface to receive 3D image models of their skin surface, in accordance with the techniques of the present disclosure. For example, FIGS. 5A-5C illustrate example images 130a, 130b, and 130c that may be imaged and analyzed by the user mobile device 202 and dermatological imaging device 110 combination to generate 3D image models of a user's skin surface. Each of these images may be collected/aggregated at the user mobile device 202 and may be analyzed by, and/or used to train, a 3D image modeling algorithm (e.g., 3D image modeling algorithm 108). In some embodiments, the skin surface images may be collected or aggregated at imaging server(s) 102 and may be analyzed by, and/or used to train, the 3D image modeling algorithm (e.g., an AI model such as a machine learning image modeling model, as described herein).

Each image representing the example regions 130a, 130b, 130c may comprise pixel data 502ap, 502bp, and 502cp (e.g., RGB data) representing feature data and corresponding to each of the particular attributes of the respective skin surfaces within the respective image. Generally, as described herein, the pixel data 502ap, 502 bp, 502cp comprises points or squares of data within an image, where each point or square represents a single pixel (e.g., pixels 502ap1, 502ap2, 502bp1, 502bp2, 502cp1, and 502cp2) within an image. Each pixel may be a specific location within an image. In addition, each pixel may have a specific color (or lack thereof). Pixel color may be determined by a color format and related channel data associated with a given pixel. For example, a popular color format includes the red-green-blue (RGB) format having red, green, and blue channels. That is, in the RGB format, data of a pixel is represented by three numerical RGB components (Red, Green, Blue), that may be referred to as a channel data, to manipulate the color of pixel's area within the image. In some implementations, the three RGB components may be represented as three 8-bit numbers for each pixel. Three 8-bit bytes (one byte for each of RGB) is used to generate 24-bit color. Each 8-bit RGB component can have 256 possible values, ranging from 0 to 255 (i.e., in the base 2 binary system, an 8-bit byte can contain one of 256 numeric values ranging from 0 to 255). This channel data (R, G, and B) can be assigned a value from 0 255 and be used to set the pixel's color. For example, three values like (250, 165, 0), meaning (Red=250, Green=165, Blue=0), can denote one Orange pixel. As a further example, (Red=255, Green=255, Blue=0) means Red and Green, each fully saturated (255 is as bright as 8 bits can be), with no Blue (zero), with the resulting color being Yellow. As a still further example, the color black has an RGB value of (Red=0, Green=0, Blue=0) and white has an RGB value of (Red=255, Green=255, Blue=255). Gray has the property of having equal or similar RGB values. So (Red=220, Green=220, Blue=220) is a light gray (near white), and (Red=40, Green=40, Blue=40) is a dark gray (near black).

In this way, the composite of three RGB values creates the final color for a given pixel. With a 24-bit RGB color image using 3 bytes there can be 256 shades of red, and 256 shades of green, and 256 shades of blue. This provides 256×256×256, i.e., 16.7 million possible combinations or colors for 24-bit RGB color images. In this manner, the pixel's RGB data value shows how much of each of Red, and Green, and Blue the pixel is comprised of. The three colors and intensity levels are combined at that image pixel, i.e., at that pixel location on a display screen, to illuminate a display screen at that location with that color. It is to be understood, however, that other bit sizes, having fewer or more bits, e.g., 10-bits, may be used to result in fewer or more overall colors and ranges. For example, the user mobile device 202 may analyze the captured images in grayscale, instead of an RGB color space.

As a whole, the various pixels, positioned together in a grid pattern, form a digital image (e.g., images 130a, 130b, and/or 130c). A single digital image can comprise thousands or millions of pixels. Images can be captured, generated, stored, and/or transmitted in a number of formats, such as JPEG, TIFF, PNG and GIF. These formats use pixels to store and represent the image.

FIG. 5A illustrates an example image 130a and its related pixel data (e.g., pixel data 502ap) that may be used for training and/or implementing a 3D image modeling algorithm (e.g., 3D image modeling algorithm 108), in accordance with various embodiments disclosed herein. The example image 130a illustrates a portion of a user's skin surface featuring an acne lesion (e.g., the user's facial area). In various embodiments, the user may capture an image for analysis by the user mobile device 202 of at least one of the user's face, the user's cheek, the user's neck, the user's jaw, the user's head, the user's groin, the user's underarm, the user's chest, the user's back, the user's leg, the user's arm, the user's abdomen, the user's feet, and/or any other suitable area of the user's body or combinations thereof. The example image 130a may represent, for example, a user attempting to track the formation and elimination of an acne lesion over time using the user mobile device 202 and dermatological imaging device 110 combination, as discussed herein.

The image 130a is comprised of pixel data 502ap including, for example, pixels 502ap1 and 502ap2. Pixel 502ap1 may be a relatively dark pixel (e.g., a pixel with low R, G, and B values) positioned in image 130a resulting from the user having a relatively low degree of skin undulation/reflectivity at the position represented by pixel 502ap1 due to, for example, abnormalities on the skin surface (e.g., an enlarged pore(s) or damaged skin cells). Pixel 502ap2 may be a relatively lighter pixel (e.g., a pixel with high R, G, and B values) positioned in image 130a resulting from the user having the acne lesion at the position represented by pixel 502ap2.

The user mobile device 202 and dermatological imaging device 110 combination may capture the image 130a under multiple angles/intensities of illumination (e.g., via LEDs 220), as part of a video sampling period and illumination sequence. Accordingly, the pixel data 502ap may include multiple darkness/lightness values for each individual pixel (e.g., 502ap1, 502ap2) corresponding to the multiple illumination angles/intensities associated with each capture of the image 130a during the video sampling period. The pixel 502ap1 may generally appear darker than the pixel 502ap2 in the image captures of the video sampling period due to the difference in features represented by the two pixels 502ap1, 502ap2. Thus, this difference in dark/light appearance and any shadows cast that are attributable to the pixel 502ap2 may, in part, cause the 3D image modeling algorithm 108 to display the pixel 502ap2 as a raised portion of the skin surface represented by the image 130a relative to the pixel 502ap1, as discussed further herein.

FIG. 5B illustrates a further example image 130b and its related pixel data (e.g., pixel data 502 bp) that may be used for training and/or implementing a 3D image modeling algorithm (e.g., 3D image modeling algorithm 108), in accordance with various embodiments disclosed herein. The example image 130b illustrates a portion of a user's skin surface including an actinic keratosis lesion (e.g., the user's hand or arm area). The example image 130b may represent, for example, the user utilizing the user mobile device 202 and dermatological imaging device 110 combination to examine/analyze the micro relief of a skin lesion formed on the user's hand.

Image 130b is comprised of pixel data, including pixel data 502 bp. Pixel data 502 bp includes a plurality of pixels including pixel 502bp1 and pixel 502bp2. Pixel 502bp1 may be a light pixel (e.g., a pixel with high R, G, and/or B values) positioned in image 130b resulting from the user having a relatively low degree of skin undulation at the position represented by pixel 502 bp1. Pixel 502bp2 may be a dark pixel (e.g., a pixel with low R, G, and B values) positioned in image 130b resulting from the user having a relatively high degree of skin undulation at the position represented by pixel 502bp2 due to, for example, the skin lesion.

The user mobile device 202 and dermatological imaging device 110 combination may capture the image 130b under multiple angles/intensities of illumination (e.g., via LEDs 220), as part of a video sampling period and illumination sequence. Accordingly, the pixel data 502 bp may include multiple darkness/lightness values for each individual pixel (e.g., 502 bp1, 502bp2) corresponding to the multiple illumination angles/intensities associated with each capture of the image 130b during the video sampling period. The pixel 502bp2 may generally appear darker than the pixel 502bp1 in the image captures of the video sampling period due to the difference in features represented by the two pixels 502bp1, 502bp2. Thus, this difference in dark/light appearance and any shadows cast on the pixel 502bp2 may, in part, cause the 3D image modeling algorithm 108 to display the pixel 502bp1 as a raised portion of the skin surface represented by the image 130b relative to the pixel 502bp2, as discussed further herein.

FIG. 5C illustrates a further example image 130c and its related pixel data (e.g., 502cp) that may be used for training and/or implementing a 3D image modeling algorithm (e.g., 3D image modeling algorithm 108), in accordance with various embodiments disclosed herein. The example image 130c illustrates a portion of a user's skin surface including a skin flare-up (e.g., the user's chest or back area) as a result of an allergic reaction the user is experiencing. The example image 130c may represent, for example, the user utilizing the user mobile device 202 and dermatological imaging device 110 combination to examine/analyze the flare-up caused by the allergic reaction, as discussed further herein.

Image 130c is comprised of pixel data, including pixel data 502cp. Pixel data 502cp includes a plurality of pixels including pixel 502cp1 and pixel 502cp2. Pixel 502cp1 may be a light-red pixel (e.g., a pixel with a relatively high R value) positioned in image 130c resulting from the user having a skin flare-up at the position represented by pixel 502cp1. Pixel 502cp2 may be a light pixel (e.g., a pixel with high R, G, and/or B values) positioned in image 130c resulting from user 130cu having a minimal skin flare-up at the position represented by pixel 502cp2.

The user mobile device 202 and dermatological imaging device 110 combination may capture the image 130c under multiple angles/intensities of illumination (e.g., via LEDs 220), as part of a video sampling period and illumination sequence. Accordingly, the pixel data 502cp may include multiple darkness/lightness values and multiple color values for each individual pixel (e.g., 502cp1, 502cp2) corresponding to the multiple illumination angles/intensities associated with each capture of the image 130c during the video sampling period. The pixel 502cp2 may generally appear lighter and more of a neutral skin tone than the pixel 502cp1 in the image captures of the video sampling period due to the difference in features represented by the two pixels 502cp1, 502cp2. Thus, this difference in dark/light appearance, RGB color values, and any shadows cast that are attributable to the pixel 502cp2 may, in part, cause the 3D image modeling algorithm 108 to display the pixel 502cp1 as a raised, redder portion of the skin surface represented by the image 130c relative to the pixel 502cp2, as discussed further herein.

The pixel data 130ap, 130 bp, and 130cp each include various remaining pixels including remaining portions of the user's skin surface area featuring varying lightness/darkness values and color values. The pixel data 130ap, 130 bp, and 130cp each further include pixels representing further features including the undulations of the user's skin due to anatomical features of the user's skin surface and other features as shown in FIGS. 5A-5C.

It is to be understood that each of the images represented in FIGS. 5A-5C may arrive and be processed in accordance with a 3D image modeling algorithm (e.g., 3D image modeling algorithm 108), as described further herein, in real-time and/or near real-time. For example, a user may capture image 130c as the allergic reaction is taking place, and the 3D image modeling algorithm may provide feedback, recommendations, and/or other comments in real-time or near real-time.

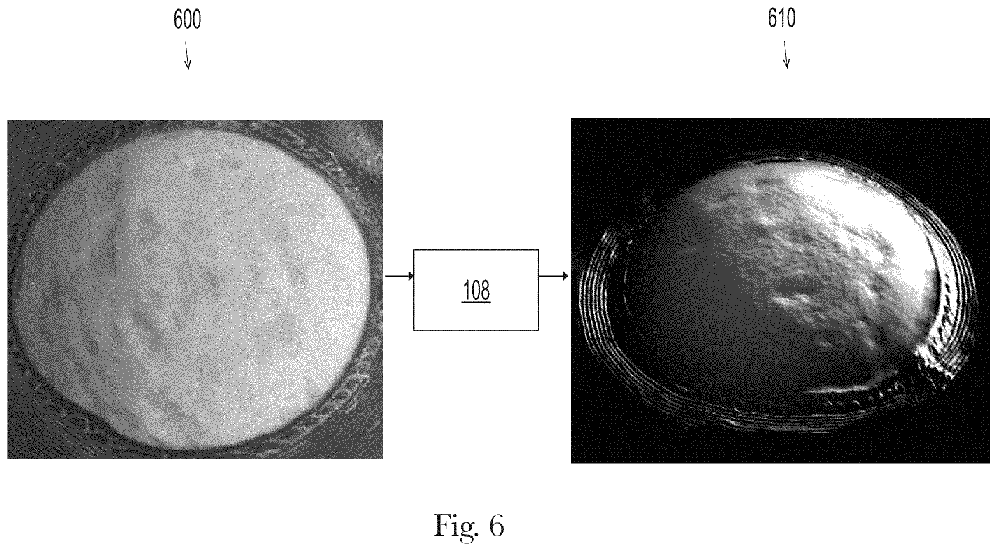

In any event, when the images are captured by the user mobile device 202 and dermatological imaging device 110 combination, the images may be processed by the 3D image modeling algorithm 108 stored at the user mobile device 202 (e.g., as part of a mobile application). FIG. 6 illustrates an example workflow of the 3D image modeling algorithm 108 using an input skin surface image 600 to generate a 3D image model 610 defining a topographic representation of the skin surface. Generally, the 3D image modeling algorithm 108 may analyze pixel values of multiple skin surface images (e.g., similar to the input skin surface image 600) to construct the 3D image model 610.

More specifically, the 3D image modeling algorithm 108 may estimate the 3D image model 610 by utilizing pixel values to solve the photometric stereo equation, as given by:

$${I}_{i} = {ρ}_{i} \frac{{\hat{N}}_{i} \cdot ( {\vec{L}}_{j} - {\vec{P}}_{i} )}{{‖ {\vec{L}}_{j} - {\vec{P}}_{i} ‖}^{q}} , \qquad ( 1 )$$

where Nᵢ is the normal at the i^{th} 3D point \vec{P}ᵢ on the skin surface, ρᵢ is the Albedo, \vec{L}ⱼ is the 3D location of the j^{th} light source (e.g., LEDs 220) and q is the light attenuation factor. The 3D image modeling algorithm 108 may, for example, integrate a differential light contribution from a probabilistic cone of illumination for each pixel and use an observed intensity for each pixel to correct the estimated normals from equation (1). With the corrected normals, the 3D image modeling algorithm 108 may generate the 3D image model 610 using, for example, a depth from gradient algorithm.

Estimating the 3D image model 610 may be highly dependent on the skin type (e.g., skin color, skin surface area, etc.) corresponding to the skin surface represented in the captured images. Advantageously, the 3D image modeling algorithm 108 may automatically determine a skin type corresponding to the skin surface represented in the captured images by iteratively estimating the normals in accordance with equation (1). The 3D image modeling algorithm 108 may also balance the pixel intensities across the captured images to facilitate the determination of skin type, in view of the estimated normals for each pixel.

Moreover, the 3D image modeling algorithm 108 may estimate the probabilistic cone of illumination for a particular captured image when generating the 3D image model 610. Generally, when a light source illuminating an imaged planar surface is at infinity, the light rays incident to the planar surface are assumed to be parallel, and all points on the planar surface are illuminated with equal intensity. However, when the light source is much closer to the surface (e.g., within 35 mm or less), the light rays incident to the planar surface form a cone. As a result, points on the planar surface that are close to the light source are brighter than points on the planar surface that are further away from the light source. Accordingly, the 3D image modeling algorithm 108 may estimate the probabilistic cone of illumination for a captured image using the captured image in conjunction with the known dimensional parameters describing the user mobile device 202 and dermatological imaging device 110 combination (e.g., 3D LED 220 position, distance from LEDs 220 to ROI, distance from camera 212 to ROI, etc.).

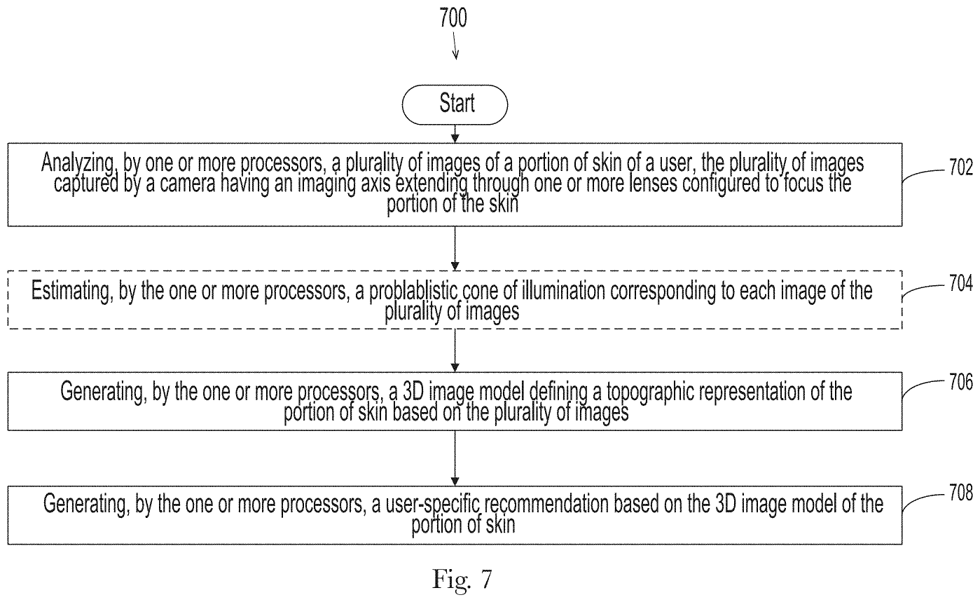

FIG. 7 illustrates a diagram of a dermatological imaging method 700 of analyzing pixel data of an image (e.g., images 130a, 130b, and/or 130c) of a user's skin surface for generating three-dimensional (3D) image models of skin surfaces, in accordance with various embodiments disclosed herein. Images, as described herein, are generally pixel images as captured by a digital camera (e.g., the camera 212 of user mobile device 202). In some embodiments, an image may comprise or refer to a plurality of images such as a plurality of images (e.g., frames) as collected using a digital video camera. Frames comprise consecutive images defining motion, and can comprise a movie, a video, or the like.

At block 702, the method 700 comprises analyzing, by one or more processors, images of a portion of skin of a user, where the images are captured by a camera (e.g., camera 212) having an imaging axis extending through one or more lenses (e.g., lens set 216) configured to focus the portion of skin. Each image may be illuminated by a different subset of LEDs (e.g., LEDs 220) that are configured to be positioned approximately at a perimeter of the portion of skin. For example, the images may represent a respective user's acne lesion (e.g., as illustrated in FIG. 5A), a respective user's actinic keratosis lesion (e.g., as illustrated in FIG. 5B), a respective user's allergic flare-up (e.g., as illustrated in FIG. 5C), and/or a respective user's skin condition (or lack thereof) of any kind located on a respective user's head, a respective user's groin, a respective user's underarm, a respective user's chest, a respective user's back, a respective user's leg, a respective user's arm, a respective user's abdomen, a respective user's feet, and/or any other suitable area of a respective user's body or combinations thereof.

In some embodiments, a subset of LEDs may illuminate the portion of skin at a first illumination intensity, and a different subset of LEDs may illuminate the portion of skin at a second illumination intensity that is different from the first illumination intensity. For example, a first LED may illuminate the portion of skin at a first wattage, and a second LED may illuminate the portion of skin at a second wattage. In this example, the second wattage may be twice the value of the first wattage, such that the second LED illuminates the portion of skin at twice the intensity of the first LED.

Further, in some embodiments, the illumination provided by each different subset of LEDs may illuminate the portion of skin from a different illumination angle. For example, assume that a parallel line (e.g., a “normal” line) to the orientation of the user mobile device 202 extending vertically in both directions from the center of the ROI defines a zero-degree illumination angle. Accordingly, a first LED may illuminate the portion of skin from a first illumination angle of ninety degrees from the normal line, and a second LED may illuminate the portion of skin from a second illumination angle of thirty degrees from the normal line. In this example, a first captured image that was illuminated by the first LED from the first illumination angle may include different shadows than a second captured image that was illuminated by the second LED from the second illumination angle. As a result, each image captured by the user mobile device 202 and dermatological imaging device 110 combination may feature a different set of shadows cast on the portion of skin as a result of illumination from a different illumination angle.

Additionally, in some embodiments, the user mobile device 202 (e.g., via a mobile application) may calibrate the camera 212 using a random sampling consensus algorithm prior to analyzing the captured images. The random sampling consensus algorithm may be configured to select ideal images from a video capture sequence of a calibration plate. As referenced herein, the video capture sequence may collectively refer to the “video sampling period” and the “illumination sequence” described herein. For example, the user mobile device 202 may utilize a video capture sequence to calibrate the camera 212, LEDs 220, and/or any other suitable hardware. Further, the user mobile device 202 may utilize a video capture sequence to generate a 3D image model of a user's skin surface. In these embodiments, the user mobile device 202 may also calibrate the LEDs 220 by path tracing light rays reflected from multiple reflective objects (e.g., objects 312).

In some embodiments, the user mobile device 202 may capture the images at a short imaging distance. For example, the short imaging distance may be 35 mm or less, such that the distance between the camera and the ROI (e.g., as defined by the aperture 218) is less than or equal to 35 mm.

In some embodiments, the camera 212 may capture the images during a video capture sequence, and each different subset of LEDs 220 may be sequentially activated and sequentially deactivated during the video capture sequence (e.g., as part of the illumination sequence). Further in these embodiments, the 3D image modeling algorithm 108 may compute a mean pixel intensity for each image, and align each image with a respective maximum mean pixel intensity. For example, and as previously mentioned, if the dermatological imaging device 110 includes twenty-one LEDs 220, then the 3D image modeling algorithm 108 may designate twenty-one images as maximum mean pixel intensity images. Moreover, the LEDs 220 and the camera 212 may be asynchronously controlled by the user mobile device 202 (e.g., via the mobile application) during the video capture sequence.

At optional block 704, the method 700 may comprise the 3D image modeling algorithm 108 estimating a probabilistic cone of illumination corresponding to each image. For example, and as previously mentioned, the 3D image modeling algorithm 108 may utilize processors of the user mobile device 202 (e.g., any of user computing devices 111c1-111c3 and/or 112c1-112c3) and/or the imaging server(s) 102 to estimate the probabilistic cone of illumination for captured images. The probabilistic cone may represent the estimated incident illumination from an LED 220 on the ROI during the image capture.

At block 706, the method 700 may comprise generating, by one or more processors, a 3D image model (e.g., 3D image model 610) defining a topographic representation of the portion of skin based on the captured images. The 3D image model may be generated by, for example, the 3D image modeling algorithm 108. In some embodiments, the 3D image modeling algorithm 108 may compare the 3D image model to another 3D image model that defines another topographic representation of a portion of skin of another user. In these embodiments, the other user may share an age or a skin condition with the user. The skin condition may include at least one of (i) skin cancer, (ii) a sun burn, (iii) acne, (iv) xerosis, (v) seborrhoea, (vi) eczema, or (vii) hives.

In some embodiments, the 3D image modeling algorithm 108 may determine that the 3D image model defines a topographic representation corresponding to skin of a set of users having a skin type class. Generally, the skin type class may correspond to any suitable characteristic of skin, such as pore size, redness, scarring, lesion count, freckle density, and/or any other suitable characteristic or combinations thereof. In further embodiments, the skin type class may correspond to a color of skin.

In various embodiments, the 3D image modeling algorithm 108 is an artificial intelligence (AI) based model trained with at least one AI algorithm. Training of the 3D image modeling algorithm 108 involves image analysis of the training images to configure weights of the 3D image modeling algorithm 108, used to predict and/or classify future images. For example, in various embodiments herein, generation of the 3D image modeling algorithm 108 involves training the 3D image modeling algorithm 108 with the plurality of training images of a plurality of users, where each of the training images comprise pixel data of a respective user's skin surface. In some embodiments, one or more processors of a server or a cloud-based computing platform (e.g., imaging server(s) 102) may receive the plurality of training images of the plurality of users via a computer network (e.g., computer network 120). In such embodiments, the server and/or the cloud-based computing platform may train the 3D image modeling algorithm 108 with the pixel data of the plurality of training images.

In various embodiments, a machine learning imaging model, as described herein (e.g., 3D image modeling algorithm 108), may be trained using a supervised or unsupervised machine learning program or algorithm. The machine learning program or algorithm may employ a neural network, which may be a convolutional neural network, a deep learning neural network, or a combined learning module or program that learns in two or more features or feature datasets (e.g., pixel data) in a particular areas of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. In some embodiments, the artificial intelligence and/or machine learning based algorithms may be included as a library or package executed on imaging server(s) 102. For example, libraries may include the TENSORFLOW based library, the PYTORCH library, and/or the SCIKIT-LEARN Python library.

Machine learning may involve identifying and recognizing patterns in existing data (such as training a model based on pixel data within images having pixel data of a respective user's skin surface) in order to facilitate making predictions or identification for subsequent data (such as using the model on new pixel data of a new user in order to generate a 3D image model of the new user's skin surface).

Machine learning model(s), such as the 3D image modeling algorithm 108 described herein for some embodiments, may be created and trained based upon example data (e.g., “training data” and related pixel data) inputs or data (which may be termed “features” and “labels”) in order to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated. The disclosures herein may use one or both of such supervised or unsupervised machine learning techniques.

Image analysis may include training a machine learning based algorithm (e.g., the 3D image modeling algorithm 108) on pixel data of images of one or more user's skin surface. Additionally, or alternatively, image analysis may include using a machine learning imaging model, as previously trained, to generate, based on the pixel data (e.g., including their RGB values) of the one or more images of the user(s), a 3D image model of the specific user's skin surface. The weights of the model may be trained via analysis of various RGB values of user pixels of a given image. For example, dark or low RGB values (e.g., a pixel with values R=25, G=28, B=31) may indicate a relatively low-lying area of the user's skin surface. A red toned RGB value (e.g., a pixel with values R=215, G=90, B=85) may indicate irritated skin. A lighter RGB value (e.g., a pixel with R=181, G=170, and B=191) may indicate a relatively elevated area of the user's skin (e.g., such as an acne lesion). In this manner, pixel data (e.g., detailing one or more features of a user's skin surface) of 10,000s training images may be used to train or use a machine learning imaging algorithm to generate a 3D image model of a specific user's skin surface.

At block 708, the method 700 comprises generating, by the one or more processors (e.g., user mobile device 202), a user-specific recommendation based upon the 3D image model of the user's portion of skin. For example, the user-specific recommendation may be a user-specific product recommendation for a manufactured product. Accordingly, the manufactured product may be designed to address at least one feature identifiable within the pixel data of the user's portion of skin. In some embodiments, the user-specific recommendation recommends that the user apply a product to the portion of skin or seek medical advice regarding the portion of skin. If, for example, the 3D image modeling algorithm 108 determines that the user's portion of skin includes characteristics indicative of skin cancer, the 3D image modeling algorithm 108 may generate a user-specific recommendation advising the user to seek immediate medical attention.

In some embodiments, the user mobile device 202 may capture a second plurality of images of the user's portion of skin. The camera 212 of the user mobile device 202 may capture the images, and each image of the second plurality may be illuminated by a different subset of the LEDs 220. The 3D image modeling algorithm 108 may then generate, based on the second plurality of images, a second 3D image model that defines a second topographic representation of the portion of skin. Moreover, the 3D image modeling algorithm 108 may compare the first 3D image model to the second 3D image model to generate the user-specific recommendation. For example, a user may initially capture a first set of images of a skin surface including an acne lesion (e.g., as illustrated in FIG. 5A). Several days later, the user may capture a second set of images of the skin surface containing the acne lesion, and the 3D image modeling algorithm may calculate a volume/height reduction of the acne lesion over the several days by comparing the first and second sets of images. As another example, the 3D image modeling algorithm 108 may compare the first and second sets of images to track roughness measurements of the user's portion of skin, and may further be applied to track the development of wrinkles, moles, etc. over time. Other examples may include tracking/studying the micro relief in skin lesions (e.g., the actinic keratosis lesion illustrated in FIG. 5B), skin flare-ups caused by allergic reactions (e.g., the allergic flare-up illustrated in FIG. 5C) to measure the efficacy of antihistamines in quelling the reactions, scars and scarring tissues to determine the effectiveness of medication intended to heal the skin surface, chapped lips/skin flakes to measure the effectiveness of lip balms, and/or any other suitable purpose or combinations thereof.

In some embodiments, the user mobile device 202 may execute a mobile application that comprises instructions that are executable by one or more processors of the user mobile device 202. The mobile application may be stored on a non-transitory computer-readable medium of the user mobile device 202. The instructions, when executed by the one or more processors, may cause the one or more processors to render, on a display screen of the user mobile device 202, the 3D image model. The instructions may further cause the one or more processors to render an output textually describing or graphically illustrating a feature of the 3D image model on the display screen.

In some embodiments, the 3D image modeling algorithm 108 may be trained with a plurality of 3D image models each depicting a topographic representation of a portion of skin of a respective user. The 3D image modeling algorithm 108 may be trained to generate the user-specific recommendation by analyzing the 3D image model (e.g., the 3D image model 610) of the portion of skin. Moreover, computing instructions stored on the user mobile device 202, when executed by one or more processors of the device 202, may cause the one or more processors to analyze, with the 3D image modeling algorithm 108, the 3D image model to generate the user-specific recommendation based on the 3D image model of the portion of skin. The user mobile device 202 may additionally include a display screen configured to receive the 3D image model and to render the 3D image model in real-time or near real-time upon or after capture of the plurality of images by the camera 212.

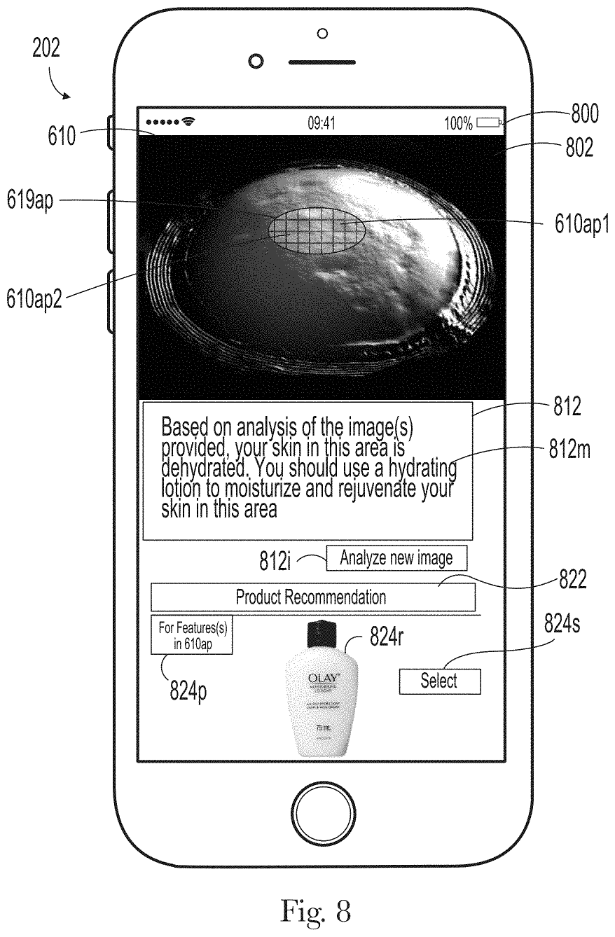

As an example of the graphical display(s), FIG. 8 illustrates an example user interface 802 as rendered on a display screen 800 of a user mobile device 202, in accordance with various embodiments disclosed herein. For example, as shown in the example of FIG. 8, the user interface 802 may be implemented or rendered via an application (app) executing on the user mobile device 202.

As shown in the example of FIG. 8, the user interface 802 may be implemented or rendered via a native app executing on the user mobile device 202. In the example of FIG. 8, the user mobile device 202 is a user computing device as described for FIGS. 1 and 2, e.g., where the user computing device 111c1 and the user mobile device 202 are illustrated as APPLE iPhones that implement the APPLE iOS operating system, and the user mobile device 202 has a display screen 800. User mobile device 202 may execute one or more native applications (apps) on its operating system. Such native apps may be implemented or coded (e.g., as computing instructions) in a computing language (e.g., SWIFT) executable by the user computing device operating system (e.g., APPLE iOS) by the processor of user mobile device 202. Additionally, or alternatively, the user interface 802 may be implemented or rendered via a web interface, such as via a web browser application, e.g., Safari and/or Google Chrome app(s), or other such web browser or the like.

As shown in the example of FIG. 8, the user interface 802 comprises a graphical representation (e.g., 3D image model 610) of the user's skin. The graphical representation may be the 3D image model 610 of the user's skin surface as generated by the 3D image modeling algorithm 108, as described herein. In the example of FIG. 8, the 3D image model 610 of the user's skin surface may be annotated with one or more graphics (e.g., area of pixel data 610ap), textual rendering, and/or any other suitable rendering or combinations thereof corresponding to the topographic representation of the user's skin surface. It is to be understood that other graphical/textual rendering types or values are contemplated herein, where textual rendering types or values may be rendered, for example, as a roughness measurement of the indicated portion of skin (e.g., at pixel 610ap2), a change in volume/height of an acne lesion (e.g., at pixel 610ap1), or the like. Additionally, or alternatively, color values may be used and/or overlaid on a graphical representation shown on the user interface 802 (e.g., 3D image model 610) to indicate topographic features of the user's skin surface (e.g., heat-mapping detailing changes in topographical features over time).

Other graphical overlays may include, for example, a heat mapping, where a specific color scheme overlaid onto the 3D image model 610 indicates a magnitude or a direction of topographical feature movement over time and/or dimensional differences between features within the 3D image model 610 (e.g., height differences between features). The 3D image model 610 may also include textual overlays configured to annotate the relative magnitudes and/or directions indicated by arrow(s) and/or other graphical overlay(s). For example, the 3D image model 610 may include text such as “Sunburn,” “Acne Lesion,” “Mole,” “Scar Tissue,” etc. to describe the features indicated by arrows and/or other graphical representations. Additionally or alternatively, the 3D image model 610 may include a percentage scale or other numerical indicator to supplement the arrows and/or other graphical indicators. For example, the 3D image model 610 may include skin roughness values from 0% to 100%, where 0% represents the least skin roughness for a particular skin surface portion and 100% represents the maximum skin roughness for a particular skin surface portion. Values can range across this map where a skin roughness value of 67% represents one or more pixels detected within the 3D image model 610 that has a higher skin roughness value than a skin roughness value of 10% as detected for one or more different pixels within the same 3D image model 610 or a different 3D image model (of the same or different user and/or portion of skin). Moreover, the percentage scale or other numerical indicators may be used internally when the 3D image modeling algorithm 108 determines the size and/or direction of the graphical indicators, textual indicators, and/or other indicators or combinations thereof.

For example, the area of pixel data 610ap may be annotated or overlaid on top of the 3D image model 610 to highlight the area or feature(s) identified within the pixel data (e.g., feature data and/or raw pixel data) by the 3D image modeling algorithm 108. In the example of FIG. 8, the feature(s) identified within the area of pixel data 610ap may include skin surface abnormalities (e.g., moles, acne lesions, etc.), irritation of the skin (e.g., allergic reactions), skin type (e.g., estimated age values), skin tone, and other features shown in the area of pixel data 610ap. In various embodiments, the pixels identified as specific features within the pixel data 610ap (e.g., pixel 610ap1 and pixel 610ap2) may be highlighted or otherwise annotated when rendered.

User interface 802 may also include or render a user-specific recommendation 812. In the embodiment of FIG. 8, the user-specific recommendation 812 comprises a message 812m to the user designed to address a feature identifiable within the pixel data (e.g., pixel data 610ap) of the user's skin surface. As shown in the example of FIG. 8, the message 812m includes a product recommendation for the user to apply a hydrating lotion to moisturize and rejuvenate their skin, based on an analysis of the 3D image modeling algorithm 108 that indicated the user's skin surface is dehydrated. The product recommendation may be correlated to the identified feature within the pixel data (e.g., hydrating lotion to alleviate skin dehydration), and the user mobile device 202 may be instructed to output the product recommendation when the feature (e.g., skin dehydration, sunburn, etc.) is identified. As previously mentioned, the user mobile device 202 may include a recommendation for the user to seek medical treatment/advice in cases where the 3D image modeling algorithm 108 identifies features within the pixel data that are indicative of medical conditions for which the user may require/desire a medical opinion (e.g., skin cancer).

The user interface 802 may also include or render a section for a product recommendation 822 for a manufactured product 824r (e.g., hydrating/moisturizing lotion, as described above). The product recommendation 822 generally corresponds to the user-specific recommendation 12, as described above. For example, in the example of FIG. 8, the user-specific recommendation 812 may be displayed on the display screen 800 of the user mobile device 202 with instructions (e.g., message 812m) for treating, with the manufactured product (manufactured product 824r (e.g., hydrating/moisturizing lotion)) at least one feature (e.g., skin dehydration at pixel 610ap1, 610ap2) identifiable in the pixel data (e.g., pixel data 610ap) of the user's skin surface.

As shown in FIG. 8, the user interface 802 presents a recommendation for a product (e.g., manufactured product 824r (e.g., hydrating/moisturizing lotion)) based on the user-specific recommendation 812. In the example of FIG. 8, the output or analysis of image(s) (e.g. skin surface image 600) using the 3D image modeling algorithm 108, may be used to generate or identify recommendations for corresponding product(s). Such recommendations may include products such as hydrating/moisturizing lotion, exfoliator, sunscreen, cleanser, shaving gel, or the like to address the feature detected within the pixel data by the 3D image modeling algorithm 108. In the example of FIG. 4, the user interface 802 renders or provides a recommended product (e.g., manufactured product 824r), as determined by the 3D image modeling algorithm 108, and its related image analysis of the 3D image model 610 and its pixel data and various features. In the example of FIG. 8, this is indicated and annotated (824p) on the user interface 802.

The user interface 802 may further include a selectable UI button 824s to allow the user to select for purchase or shipment the corresponding product (e.g., manufactured product 824r). In some embodiments, selection of the selectable UI button 824s may cause the recommended product(s) to be shipped to the user and/or may notify a third party that the user is interested in the product(s). For example, either the user mobile device 202 and/or the imaging server(s) 102 may initiate, based on the user-specific recommendation 812, the manufactured product 824r (e.g., hydrating/moisturizing lotion) for shipment to the user. In such embodiments, the product may be packaged and shipped to the user.

In various embodiments, the graphical representation (e.g., 3D image model 610), with graphical annotations (e.g., area of pixel data 610ap), and the user-specific recommendation 812 may be transmitted, via the computer network (e.g., from an imaging server 102 and/or one or more processors) to the user mobile device 202, for rendering on the display screen 800. In other embodiments, no transmission to the imaging server(s) 102 of the user's specific image occurs, where the user-specific recommendation (and/or product specific recommendation) may instead be generated locally, by the 3D image modeling algorithm 108 executing and/or implemented on the user mobile device 202 and rendered, by a processor of the mobile device, on the display screen 800 of the user mobile device 202.

In some embodiments, as shown in the example of FIG. 8, the user may select selectable button 812i for reanalyzing (e.g., either locally at user mobile device 202 or remotely at imaging server(s) 102) a new image. Selectable button 812i may cause the user interface 802 to prompt the user to position the user mobile device 202 and dermatological imaging device 110 combination over the user's skin surface to capture a new image and/or for the user to select a new image for upload. The user mobile device 202 and/or the imaging server(s) 102 may receive the new image of the user before, during, and/or after performing some or all of the treatment options/suggestions presented in the user-specific recommendation 812. The new image (e.g., just like skin surface image 600) may comprise pixel data of the user's skin surface. The 3D image modeling algorithm 108, executing on the memory of the user mobile device 202, may analyze the new image captured by the user mobile device 202 and dermatological imaging device 110 combination to generate a new 3D image model of the user's skin surface. The user mobile device 202 may generate, based on the new 3D image model, a new user-specific recommendation or comment regarding a feature identifiable within the pixel data of the new 3D image model. For example, the new user-specific recommendation may include a new graphical representation including graphics and/or text. The new user-specific recommendation may include additional recommendations, e.g., that the user should continue to apply the recommended product to reduce puffiness associated with a portion of the skin surface, the user should utilize the recommended product to eliminate any allergic flare-ups, the user should apply sunscreen before exposing the skin surface to sunlight to avoid worsening the current sunburn, etc. A comment may include that the user has corrected the at least one feature identifiable within the pixel data (e.g., the user has little or no skin irritation after applying the recommended product).

In some embodiments, the new user-specific recommendation or comment may be transmitted via the computer network to the user mobile device 202 of the user for rendering on the display screen 800 of the user mobile device 202. In other embodiments, no transmission to the imaging server(s) 102 of the user's new image occurs, where the new user-specific recommendation (and/or product specific recommendation) may instead be generated locally, by the 3D image modeling algorithm 108 executing and/or implemented on the user mobile device 202 and rendered, by a processor of the user mobile device 202, on a display screen 800 of the user mobile device 202.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “35 mm” is intended to mean “about 35 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dermatological imaging system configured to generate three-dimensional (3D) image models of skin surfaces, comprising:
a dermatological imaging device comprising:
a plurality of light-emitting diodes (LEDs) configured to be positioned at a perimeter of a portion of skin of a user, and
one or more lenses configured to focus the portion of skin; and
a computer application (app) comprising computing instructions that, when executed on a processor, cause the processor to:
analyze a plurality of images of the portion of skin, the plurality of images captured by a camera having an imaging axis extending through the one or more lenses, wherein each image of the plurality of images is illuminated by a different subset of the plurality of LEDs, and
generate, based on the plurality of images, a 3D image model defining a topographic representation of the portion of skin.

2. The system of claim 1, wherein a user-specific recommendation is generated based on the 3D image model of the portion of skin.

3. The system of claim 2, wherein the plurality of images is a first plurality of images, the 3D image model is a first 3D image model, the topographic representation of the portion of skin is a first topographic representation of the portion of skin, and generating the user-specific recommendation based on the 3D image model of the portion of skin further comprises the computer application comprising computing instructions that, when executed on the processor, further cause the processor to:
analyze a second plurality of images of the portion of skin, the second plurality of images captured by the camera, wherein each image of the second plurality of images is illuminated by a different subset of the plurality of LEDs;
generate, based on the second plurality of images, a second 3D image model defining a second topographic representation of the portion of skin; and
compare the first 3D image model to the second 3D image model to generate the user-specific recommendation.

4. The system of claim 1, wherein the illumination provided by each different subset of the plurality of LEDs illuminates the portion of skin from a different illumination angle, and each image of the plurality of images features a set of different shadows cast on the portion of skin as a result of the illumination from the different illumination angle.

5. The system of claim 1, wherein the computer application comprises computing instructions that, when executed on a processor, further cause the processor to: compare the 3D image model to at least one other 3D image model that defines another topographic representation of a portion of skin of another user, wherein the another user shares an age or a skin condition with the user.

6. The system of claim 5, wherein the skin condition comprises at least one of (i) skin cancer, (ii) a sun burn, (iii) acne, (iv) xerosis, (v) seborrhoea, (vi) eczema, or (vii) hives.

7. The system of claim 1, wherein the computer application comprises computing instructions that, when executed on a processor, further cause the processor to: determine that the 3D image model defines a topographic representation corresponding to skin of a set of users having a skin type class.

8. The system of claim 1, wherein each of the plurality of images are captured by the camera at a short imaging distance.

9. The system of claim 8, wherein the short imaging distance is less than or equal to 35 mm.

10. The system of claim 1, wherein the camera captures the plurality of images during a video capture sequence, each different subset of the plurality of LEDs is sequentially activated and sequentially deactivated during the video capture sequence, and the computing instructions, when executed by the one or more processors, further cause the one or more processors to:
compute a mean pixel intensity for each image of the plurality of images; and
align each of the plurality of images with a respective maximum mean pixel intensity.

11. The system of claim 10, wherein the plurality of LEDs and the camera are asynchronously controlled by the computer application during the video capture sequence.

12. The system of claim 1 wherein the computer application is a mobile application (app) configured to operate on a mobile device that is communicatively coupled to the dermatological imaging device, wherein the mobile app comprises computing instructions executable by one or more processors of the mobile device, and stored on a non-transitory computer-readable medium of the mobile device, wherein the computing instructions, when executed by the one or more processors, cause the one or more processors to render, on a display screen of the mobile device, the 3D image model.

13. The system of claim 12, wherein the computing instructions, when executed by the one or more processors, cause the one or more processors to render, on the display screen of the mobile device, an output textually describing or graphically illustrating a feature of the 3D image model.

14. The system of claim 1 further comprising:
one or more processors;
one or more memories communicatively coupled to the one or more processors;
an imaging model trained with a plurality of 3D image models each depicting a topographic representation of a portion of skin of a respective user, the imaging model trained to generate the user-specific recommendation by analyzing the 3D image model of the portion of skin; and
computing instructions executable by the one or more processors, and stored on the one or more memories, wherein the computing instructions, when executed by the one or more processors, cause the one or more processors to analyze, with the imaging model, the 3D image model to generate the user-specific recommendation based on the 3D image model of the portion of skin.

15. The system of claim 1, further comprising: a display screen configured to receive the 3D image model, wherein the display screen is configured to render the 3D image model in real-time or near real-time upon or after capture of the plurality of images by the camera.

16. A dermatological imaging method for generating three-dimensional (3D) image models of skin surfaces, comprising analyzing, by one or more processors, a plurality of images of a portion of skin of a user using the system of claim 1.

17. The method of claim 16, wherein at least one different subset of the plurality of LEDs illuminates the portion of skin at a first illumination intensity, and at least one different subset of the plurality of LEDs illuminates the portion of skin at a second illumination intensity that is different from the first illumination intensity.

18. The method of claim 16, further comprising: calibrating, by the one or more processors, the camera using a random sampling consensus algorithm configured to select one or more ideal images from a video capture sequence of a calibration plate; and calibrating, by the one or more processors, each of the plurality of LEDs by path tracing one or more light rays reflected from a plurality of reflective objects.

19. The method of claim 16, wherein analyzing the plurality of images of the portion of skin of the user further comprises: estimating, by the one or more processors, a probabilistic cone of illumination corresponding to each image of the plurality of images.

20. The dermatological imaging method of claim 16, wherein the user-specific recommendation based on the 3D image model of the portion of skin recommends that the user apply a product to the portion of skin or seek medical advice regarding the portion of skin.
